(19) United States
(12) Patent Application Publication
(10) Pub. No.: US 2022/0031712 A1
(43) Pub. Date: Feb. 3, 2022

(54) PREPARATION OF A PHARMACEUTICAL COMPOSITION OF OLODATEROL AND BUDESONIDE

(71) Applicants: Cai Gu Huang, Shrewsbury, MA (US); Liang Li, Shanghai (CN)

(72) Inventors: Cai Gu Huang, Shrewsbury, MA (US); Liang Li, Shanghai (CN)

(73) Assignee: Cai Gu Huang, Shrewsbury, MA (US)

(21) Appl. No.: 17/354,140

(22) Filed: Jun. 22, 2021

Related U.S. Application Data
(60) Provisional application No. 63/043,094, filed on Jun. 23, 2020.

(51) Int. Cl. A61K 31/58; A61K 31/538; A61K 9/00; A61K 47/40; A61K 47/18; A61K 47/12; A61K 47/02; A61K 9/08

(52) U.S. Cl. CPC A61K 31/58; A61K 31/538; A61K 9/0078; A61K 47/40; A61K 9/08; A61K 47/183; A61K 47/12; A61K 47/02; A61K 47/186

(57) ABSTRACT

The present invention is directed to a liquid pharmaceutical formulation and a method for administering the pharmaceutical formulation by nebulizing the pharmaceutical formulation with an inhaler. The propellant-free pharmaceutical formulation comprises: (a) active substances selected from budesonide and olodaterol; (b) a solvent; (c) a pharmacologically acceptable solubilizing agent; (d) a pharmacologically acceptable preservative, and (e) a pharmacologically acceptable stabilizer.

### PRIORITY STATEMENT

This application claims the benefit of the filing date of U.S. Provisional Patent Application No. 63/043,094, filed on Jun. 23, 2020, which is incorporated herein by reference in its entirety.

### BACKGROUND OF THE INVENTION

Budesonide, also known as 11β,21-dihydroxy-16α,17α-(butylidenebis(oxy))pregna-1,4-diene-3,20-dione, is a synthetic pregnane steroid and non-halogenated cyclic ketal corticosteroid, has the following chemical structure:

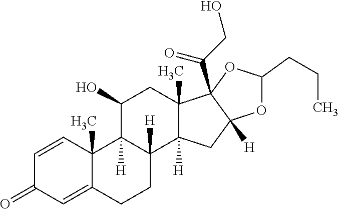

Olodaterol, chemically known as, 6-hydroxy-8-[(1R)-1-hydroxy-2-{[1-(4-methoxyphenyl)-2-methylpropan-2-yl]amino}ethyl]-3,4-dihydro-2H-1,4-benzoxazin-3-one, has the following chemical structure:

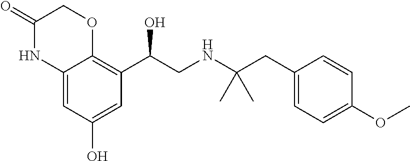 [SMILES: COc1ccc(CC(C)(C)NC[C@H](O)c2cc(O)cc3c2OCC(=O)N3)cc1]

Budesonide is a glucocorticoid with efficient local anti-inflammatory effect, it can strengthen the stability of endotheliocyte, smooth muscle cell and lysosome membrane, Immunosuppression reaction and the synthesis of reduction antibody, thus the release of the activity media such as histamine is reduced and active reduction, and can alleviate antigen-antibody in conjunction with time the enzymatic processes that excites, suppress the synthesis of bronchoconstriction material and release and alleviate the contractile response of smooth muscle.

Olodaterol is a novel, long-acting beta2-adrenergic agonist (LABA) that exerts its pharmacological effect by binding and activating beta2-adrenergic receptors located primarily in the lungs. Beta2-adrenergic receptors are membrane-bound receptors that are normally activated by endogenous epinephrine whose signaling, via a downstream L-type calcium channel interaction, mediates smooth muscle relaxation and bronchodilation. Activation of the receptor stimulates an associated G protein, which then activates adenylate cyclase, catalyzing the formation of cyclic adenosine monophosphate (cAMP) and protein kinase A (PKA). Elevation of these two molecules induces bronchodilation by relaxation of airway smooth muscles. It is by this mechanism that olodaterol is used for the treatment of chronic obstructive pulmonary disease (COPD) and the progressive airflow obstruction that is characteristic of it. Treatment with bronchodilators mitigates associated symptoms such as shortness of breath, cough, and sputum production.

These two compounds have valuable pharmacological properties. Budesonide and olodaterol can provide therapeutic benefit in the treatment of asthma or chronic obstructive pulmonary disease, including chronic bronchitis.

The present invention relates to a propellant-free inhalable formulation of budesonide and olodaterol or pharmaceutically acceptable salts or solvates thereof dissolved in water, in combination with inactive ingredients, which preferably can be administered using a nebulization inhalation device, and the propellant-free inhalable aerosols resulting therefrom. The pharmaceutical formulations of the current invention are especially suitable for administration by nebulization inhalation, which has much better lung depositions (typically up to 55-60%, even up to 85-95%), compared to methods that involve administration by drying powder inhalation.

The pharmaceutical formulations of the present invention are particularly suitable for administering the active substances by nebulization inhalation, especially for treating asthma and chronic obstructive pulmonary disease.

### SUMMARY OF THE INVENTION

The present invention relates to pharmaceutical formulations of budesonide and olodaterol and their pharmaceutically acceptable salts or solvates which can be administered by nebulization inhalation. The pharmaceutical formulations according to the invention meet high quality standards.

One aspect of the present invention is to provide an aqueous pharmaceutical formulation containing budesonide and olodaterol, which meets the high standards needed in order to be able to achieve optimum nebulization of a solution using the inhalers mentioned hereinbefore. The formulations have a storage time of some years. In one embodiment, the storage time is at least one year. In one embodiment, the storage time is at least three years.

Another aspect is to provide propellant-free formulations containing budesonide and olodaterol which is nebulized under pressure using an inhaler, which is preferably a nebulization inhaler devices, the composition delivered by the aerosol produced falling reproducibly within a specified range.

Another aspect of the invention is to provide pharmaceutical formulations comprising budesonide and olodaterol and other inactive excipients which can be administered by nebulization inhalation using ultra-sonic based or air pressure based nebulizers/inhalers. In one embodiment, the formulation has a storage time of at least 1 month. In one embodiment, the formulation has a storage time of at least 6 months. In one embodiment, the formulation has a storage time of at least one year. In one embodiment, the formulation has a storage time of at least three years.

More specifically, another aspect is to provide a stable pharmaceutical formulation of aqueous solutions containing budesonide and olodaterol and other excipients which can be administered by a nebulizer device. The formulations have substantially long term stability. In one embodiment, the formulation has a storage time of at least about 6 months at a temperature of from about 15° C. to about 25° C. In one embodiment, the formulation has a storage time of at least about 1 year at a temperature of from about 15° C. to about 25° C. ° C. In one embodiment, the formulation has a storage time of at least about 2 years at a temperature of from about 15° C. to about 25° C.

More specifically, another aspect of the current invention is to provide stable pharmaceutical formulations containing budesonide and olodaterol and other excipients which can be administered by nebulization inhalation using ultrasonic, jet or mesh nebulizers. The inventive formulations have substantially long term stability. In one embodiment, the formulation has a storage time of at least about 6 months at a temperature of from about 15° C. to about 25° C. In one embodiment, the formulation has a storage time of at least about 1 year at a temperature of from about 15° C. to about 25° C. ° C. In one embodiment, the formulation has a storage time of at least about 2 years at a temperature of from about 15° C. to about 25° C.

### DETAILED DESCRIPTION OF THE INVENTION

Administering a liquid formulation of an active agent without propellant gases using suitable inhalers achieves better delivery of the active substance to the lung. It is very important to increase the lung deposition of an active agent delivered by inhalation.

Traditional pMDI or DPI (drying powder inhalation) administration delivers only about 20-30% of a drug into the lung, resulting in a significant amount of drug being deposited on the month and throat, which can go into the stomach and cause unwanted side effects and/or secondary absorption through the oral digestive system.

Therefore, there is a need to improve drug delivery by inhalation to significantly increasing lung deposition. The soft mist or nebulization inhalation device disclosed in US20190030268 can significantly increase the lung deposition of inhalable drugs.

Those inhalers can nebulize a small amount of a liquid formulation of a drug into an aerosol that is suitable for therapeutic inhalation within a few seconds. Those inhalers are particularly suitable for administering the liquid formulations of the invention.

In one embodiment, the nebulization device containing the aqueous pharmaceutical formulation of the present invention is one in which an amount of less than about 70 microliters of the pharmaceutical formulation can be nebulized in one puff, so that the inhalable part of aerosol corresponds to the therapeutically effective quantity. In one embodiment, the nebulization device containing the aqueous pharmaceutical formulation of the present invention is one in which an amount of less than about 30 microliters of the pharmaceutical formulation can be nebulized in one puff, so that the inhalable part of aerosol corresponds to the therapeutically effective quantity. In one embodiment, the nebulization device containing the aqueous pharmaceutical formulation of the present invention is one in which an amount of less than about 15 microliters of the pharmaceutical formulation can be nebulized in one puff, so that the inhalable part of aerosol corresponds to the therapeutically effective quantity. In one embodiment, the average particle size of the aerosol formed from one puff is less than about 15 microns. In one embodiment, the average particle size of the aerosol formed from one puff is less than about 10 microns.

In one embodiment, the nebulization device containing the pharmaceutical formulation of the present invention is one in which an amount of less than about 8 milliliters of the pharmaceutical formulation can be nebulized in one puff so that the inhalable part of aerosol corresponds to the therapeutically effective quantity. In one embodiment, the nebulization device containing the pharmaceutical formulation of the present invention is one in which an amount of less than about 2 milliliters of the pharmaceutical formulation can be nebulized in one puff so that the inhalable part of aerosol corresponds to the therapeutically effective quantity. In one embodiment, the nebulization device containing the pharmaceutical formulation of the present invention is one in which an amount of less than about 1 milliliter of the pharmaceutical formulation can be nebulized in one puff so that the inhalable part of aerosol corresponds to the therapeutically effective quantity. In one embodiment, the average particle size of the aerosol formed from one puff is less than about 15 microns. In one embodiment, the average particle size of the aerosol formed from one puff is less than about 10 microns.

A device of this kind for the propellant-free administration of a metered amount of a liquid pharmaceutical composition for inhalation is described in detail in, for example, US20190030268 entitled “inhalation atomizer comprising a blocking function and a counter”.

The pharmaceutical formulation, which can be a solution, in the nebulizer is converted into aerosol destined for the lungs. The nebulizer uses high pressure to spray the pharmaceutical formulation.

The pharmaceutical solution is stored in a reservoir in this kind of inhaler. The formulation, which can be a solution, must not contain any ingredients that might interact with the inhaler to affect the pharmaceutical quality of the formulation or of the aerosol produced. In addition, the active substances in the pharmaceutical formulations are very stable when stored and can be administered directly.

In one embodiment, the formulations of the current invention for use with the inhaler described above contain additives, such as the disodium salt of edetic acid (sodium edetate), to reduce the incidence of spray anomalies and to stabilize the formulation. In one embodiment, the formulations have a minimum concentration of sodium edetate.

One aspect of the present invention is to provide a pharmaceutical formulation containing budesonide and olodaterol, which meets the high standards needed in order to achieve optimum nebulization of the formulation using a nebulizer inhaler device. In one embodiment, the formulation has a storage time of few months or years. In one embodiment, the formulation has a storage time of at least about 1 month. In one embodiment, the formulation has a storage time of at least about 6 months. In one embodiment, the formulation has a storage time of at least about one year. In one embodiment, the formulation has a storage time of at least three years.

Another aspect of the current invention is to provide propellant-free formulations, which can be solutions, containing budesonide and olodaterol, which are nebulized under pressure using an inhaler. In one embodiment, the inhaler is a nebulization inhaler. In one embodiment, the aerosol produced by the inhaler reproducibly falls within a specified range for particle size.

Another aspect of the invention is to provide an aqueous pharmaceutical formulation, which can be a solution, containing budesonide and olodaterol and other inactive excipients which can be administered by inhalation.

Any pharmaceutically acceptable salts or solvates of budesonide and olodaterol may be used for the formulation. The terms “budesonide” and “olodaterol,” as used herein, mean budesonide or a salt or solvate thereof and olodaterol or a salt or solvate thereof, respectively.

In one embodiment, the active agents are budesonide and olodaterol.

In the formulations according to the invention, the active substances are preferably selected from combinations of budesonide and olodaterol.

In one embodiment of the formulations according to the invention, budesonide and olodaterol are dissolved in a solvent. In one embodiment, the solvent comprises water. In one embodiment, the solvent is water.

In one embodiment, a therapeutically effective dose of budesonide ranges from about 1 μg to about 100 μg. In one embodiment, a therapeutically effective dose of budesonide ranges from about 5 μg to about 50 μg. In one embodiment, a therapeutically effective dose of budesonide ranges from about 10 μg to about 30 μg. In one embodiment, a therapeutically effective dose of olodaterol ranges from about 5 μg to about 500 μg. In one embodiment, a therapeutically effective dose of olodaterol ranges from about 10 μg to about 200 μg. In one embodiment, a therapeutically effective dose of olodaterol ranges from about 10 μg to about 80 μm.

In one embodiment, the concentration of budesonide in the formulation for nebulization ranges from about 1 mcg/ml to about 100 mcg/ml. In one embodiment, the concentration of budesonide in the formulation for nebulization ranges from about 5 mcg/ml to about 100 mcg/ml. In one embodiment, the concentration of budesonide in the formulation for nebulization ranges from about 10 mcg/ml to about 50 mcg/ml. In one embodiment, the concentration of olodaterol in the formulation for nebulization ranges from about 2 mcg/ml to about 500 mcg/ml. In one embodiment, the concentration of olodaterol in the formulation for nebulization ranges from about 10 mcg/ml to about 200 mcg/ml. In one embodiment, the concentration of olodaterol in the formulation for nebulization ranges from about 30 mcg/ml to about 100 mcg/ml.

In one embodiment, the formulations include a pH adjusting agent to maintain the pH of the formulation. Suitable pH adjusting agents include acids and bases. In one embodiment, the pH adjusting agent is selected from the group consisting of hydrochloric acid, citric acid and salts thereof.

Other suitable pH adjusting agents can also be used. In one embodiment, the pH adjusting agent is sodium hydroxide.

The pH influences the stability of the active substances and/or other excipients in the formulation. In one embodiment, the pH of the formulation ranges from about 2.0 to about 6.0. In one embodiment, the pH of the formulation ranges from about 3.0 to about 5.0. In one embodiment, the pH of the formulation ranges from about 3.0 to about 4.0.

In one embodiment, the formulations include a stabilizer or complexing agent. In one embodiment, the stabilizer or complexing agent is selected from edetic acid (EDTA) or one of the known salts thereof, disodium edetate or edetate disodium dihydrate. In one embodiment the formulation contains edetic acid and/or a salt thereof.

Other suitable stabilizers or complexing agents can be used in the formulations. Other suitable stabilizers or complexing agents include, but are not limited to, citric acid, edetate disodium, and edetate disodium dihydrate.

The phrase “complexing agent,” as used herein, means a molecule which is capable of entering into complex bonds. Preferably, these compounds should have the effect of complexing cations. In one embodiment, the concentration of the stabilizer or complexing agent ranges from about 0.04 mg/4 ml to about 20 mg/4 ml. In one embodiment, the concentration of the stabilizer or complexing agent ranges from about 0.2 mg/4 ml to about 8 mg/4 ml. In one embodiment, the stabilizer or complexing agent is edetate disodium dihydrate in a concentration of about 0.4 mg/4 ml.

In one embodiment, all of the ingredients of the formulation are present in solution.

The term “additives,” as used herein means any pharmacologically acceptable and therapeutically useful substance which is not an active substance, but can be formulated together with the active substances in the pharmacologically suitable solvent, in order to improve the qualities of the formulation. Preferably, these substances have no pharmacological effects or no appreciable, or at least no undesirable, pharmacological effects in the context of the desired therapy.

Suitable additives include, but are not limited to, other stabilizers, complexing agents, antioxidants, surfactants, and/or preservatives which prolong the shelf life of the finished pharmaceutical formulation, vitamins, and/or other additives known in the art.

Suitable preservatives can be added to protect the formulation from contamination with pathogenic bacteria. Suitable preservatives include, but are not limited to, benzalkonium chloride, benzoic acid, and sodium benzoate. In one embodiment, the formulation contains benzalkonium chloride as the only preservative. In one embodiment, the amount of preservative ranges from about 0.08 mg/4 ml to about 12 mg/4 ml. In one embodiment, the preservative is benzalkonium chloride in an amount of about 0.4 mg/4 ml.

In one embodiment, the formulations include a solubility enhancing agent. In one embodiment, the solubility enhancing agent is selected from the group consisting of Tween 80 and cyclodextrin derivatives. In one embodiment, the solubility enhancing agent is a cyclodextrin derivative or one of the known salts thereof. Including a solubility enhancing agent in the formulation aids the solubility of the active ingredient or other excipients. In one embodiment, the formulation contains sulfobutylether β-cyclodextrin or a salt thereof.

In one embodiment, the formulations for inhalation by nebulization include a surfactant or other solubility enhancing agent. In one embodiment, the solubility enhancing agent is selected from the group consisting of Tween 80 and cyclodextrin derivatives. In one embodiment, the solubility enhancing agent is a cyclodextrin derivative or one of the known salts thereof. In one embodiment, the solubility enhancing agent is sulfobutylether β-cyclodextrin. In one embodiment, the solubility enhancing agent is sulfobutylether β-cyclodextrin in an amount ranging from about 0.04 g/4 ml to about 1.6 g/4 ml. In one embodiment, the solubility enhancing agent is sulfobutylether β-cyclodextrin in an amount of about 0.8 g/4 ml.

Another aspect of the current invention is to provide stable pharmaceutical nebulization formulations containing budesonide and olodaterol and other excipients, which can be administered by nebulization using inhalers. In one embodiment, the formulation has substantially long term stability. In one embodiment, the formulations have a storage time of at least about 6 months at a temperature of from about 15° C. to about 25° C. In one embodiment, the formulations have a storage time of at least about 1 year at a temperature of from about 15° C. to about 25° C. In one embodiment, the formulations have a storage time of at least about 2 years at a temperature of from about 15° C. to about 25° C.

Another aspect of the current invention is to provide pharmaceutical formulations of solutions comprising budesonide and olodaterol and other inactive excipients which can be administered by nebulization inhalation using an ultra-sonic based or air pressure based nebulizer/inhaler. In one embodiment, the formulations have a storage time of few months or years. In one embodiment, the formulations have a storage time of about 1 month at a temperature of from about 15° C. to about 25° C. In one embodiment, the formulations have a storage time of about 6 months at a temperature of from about 15° C. to about 25° C. In one embodiment, the formulations have a storage time of about one year at a temperature of from about 15° C. to about 25° C. In one embodiment, the formulations have a storage time of about 2 years at a temperature of from about 15° C. to about 25° C. In one embodiment, the formulations have a storage time of about three years at a temperature of from about 15° C. to about 25° C.

Another aspect of the current invention is to provide stable pharmaceutical formulations containing budesonide and olodaterol and other excipients which can be administered by nebulization inhalation using an ultra-sonic based or air pressure based nebulizer/inhaler. In one embodiment, the formulations have substantially long term stability. In one embodiment, the formulations have a storage time of at least about 6 months at a temperature of from about 15° C. to about 25° C. In one embodiment, the formulations have a storage time of at least about 1 year at a temperature of from about 15° C. to about 25° C. In one embodiment, the formulations have a storage time of at least about 2 years at a temperature of from about 15° C. to about 25° C.

In one embodiment, the formulations include sodium chloride. In one embodiment, the concentration of sodium chloride ranges from about 0.1 g/100 ml to about 0.9 g/100 ml.

In one embodiment, the concentration of budesonide in the formulation ranges from about 1 mcg/ml to about 100 mcg/ml. In one embodiment, the concentration of budesonide in the formulation ranges from about 5 mcg/ml to about 100 mcg/ml. In one embodiment, the concentration of budesonide in the formulation ranges from about 10 mcg/ml to about 50 mcg/ml. In one embodiment, the concentration of olodaterol in the formulation ranges from about 2 mcg/ml to about 500 mcg/ml. In one embodiment, the concentration of olodaterol in the formulation ranges from about 10 mcg/ml to about 200 mcg/ml. In one embodiment, the concentration of olodaterol in the formulation ranges from about 30 mcg/ml to about 100 mcg/ml.

In one embodiment, the formulations include a surfactant or other solubility enhancing agent. In one embodiment, the surfactant or other solubility enhancing agent is selected from the group consisting of Tween 80 and cyclodextrin derivatives. In one embodiment, the surfactant or other solubility enhancing agent is a cyclodextrin derivative or one of the known salts thereof. In one embodiment, the solubility enhancing agent is sulfobutylether 3-cyclodextrin.

In one embodiment, the formulations include a surfactant or other solubility enhancing agent. In one embodiment, the surfactant or solubility enhancing agent is selected from the group consisting of Tween 80 and cyclodextrin derivatives. In one embodiment, the surfactant or solubility enhancing agent is a cyclodextrin derivatives or one of the known salts thereof. In one embodiment, the solubility enhancing agent is sulfobutylether β-cyclodextrin. In one embodiment, the sulfobutylether β-cyclodextrin is included in an amount ranging from about 5 mg/ml to about 0.4 g/ml. In one embodiment, the sulfobutylether β-cyclodextrin is included in an amount of about 0.2 g/ml.

It has surprisingly been found that sulfobutylether β-cyclodextrin not only has the effect of enhancing solubility, but has the effect of improving the stability of active ingredients.

Another aspect of the current invention is to provide stable pharmaceutical formulations containing budesonide and olodaterol and other excipients which can be administered by nebulizers. In one embodiment, the inventive formulation has substantially long-term stability. In one embodiment, the formulations have a storage time of at least about 6 months at a temperature of from about 15° C. to about 25° C. In one embodiment, the formulations have a storage time of at least about 1 year at a temperature of from about 15° C. to about 25° C. In one embodiment, the formulations have a storage time of at least about 2 years at a temperature of from about 15° C. to about 25° C.

In one embodiment, the pH of the formulation ranges from about 3 to about 6. In one embodiment, the pH of the formulation ranges from about 3 to about 5. In one embodiment, the pH of the formulation ranges from about 3 to about 4.

The formulations according to the present invention can be filled into canisters to form a highly stable formulation for use in a nebulization device. In one embodiment, the formulations exhibit substantially no particle growth, change of morphology, or precipitation. There is also no, or substantially no, problem of suspended particles being deposited on the surface of either canisters or valves, so that the formulations can be discharged from a suitable nebulization device with high dose uniformity. Suitable nebulizers include, but are not limited to, an ultrasonic nebulizer; a jet nebulizer; and a mesh nebulizer, such as Pari eFlow nebulization inhaler, or other commercially available ultrasonic nebulizer, jet nebulizer, or mesh nebulizer.

The pharmaceutical formulation in the nebulizer is converted into an aerosol destined for the lungs. The nebulizer uses high pressure to spray the pharmaceutical formulation.

Nebulizers are instruments that generate very fine particles of a liquid in a gas. As is well known, particles intended for treatment of the lower airway, i.e., the bronchial tree or the lungs, are generally less than about 10 micrometers in the largest dimension, to prevent unwanted deposition onto surfaces of the mouth and pharynx. In one embodiment, the particle size of the aerosol is less than about 10 micrometers in the largest dimension in order to achieve the desired pharmacological effect. In one embodiment, the particle size of the aerosol is less than about 5 μm in the largest dimension in order to achieve the desired pharmacological effect. In addition, particles much smaller than about 0.5 μm in the largest dimension frequently are not easily deposited at the desired location, and a large fraction of these simply will be exhaled by a patient. For these reasons, it is advantageous to produce particles having a particle size that averages between about 1 μm and about 5 μm in their largest dimension, while minimizing particles having sizes less than about 0.5 μm and greater than about 10 μm. In one embodiment, the average particle size of the aerosol ranges from about 0.5 μm to about 5 μm.

Nebulization, although used more infrequently than other drug delivery techniques, has certain advantages for special patient groups, such as young children and the very infirm. Although somewhat cumbersome equipment is needed and there may be more stringent cleaning requirements than exist for other popular delivery techniques, no particular patient skill or coordination is required: the patient merely needs to breathe normally to introduce the medication into the airway. Thus, treatment can be delivered even to an unconscious patient or an infant. Another advantage of nebulizers is that quantities of moisture are delivered to the airway, which may help to fluidize secretions and increase patient comfort.

In one embodiment, the nebulization devices used to administer the pharmaceutical formulations of the present invention are those in which an amount of less than about 8 milliliters of the pharmaceutical formulation can be nebulized in one puff, so that the inhalable part of aerosol corresponds to the therapeutically effective quantity. In one embodiment, the nebulization devices used to administer the pharmaceutical formulations of the present invention are those in which an amount of less than about 2 milliliters of the pharmaceutical formulation can be nebulized in one puff, so that the inhalable part of aerosol corresponds to the therapeutically effective quantity. In one embodiment, the nebulization devices used to administer the pharmaceutical formulations of the present invention are those in which an amount of less than about 1 milliliter of the pharmaceutical formulation can be nebulized in one puff, so that the inhalable part of aerosol corresponds to the therapeutically effective quantity. In one embodiment, the average particle size of the aerosol formed from one puff is less than about 15 microns. In one embodiment, the average particle size of the aerosol formed from one puff is less than about 10 microns.

A device of this kind for the propellant-free administration of a metered amount of a liquid pharmaceutical formulation for inhalation is described in detail in, for example, US20190030268 entitled “inhalation atomizer comprising a blocking function and a counter”.

The pharmaceutical formulation in the nebulizer is converted into an aerosol destined for the lungs. The nebulizer uses high pressure to spray the pharmaceutical formulation.

The pharmaceutical formulation is stored in a reservoir in this kind of inhalers. The formulations must not contain any ingredients which might interact with the inhaler to affect the pharmaceutical quality of the solution or of the aerosol produced. In addition, the active substances in pharmaceutical formulations are very stable when stored and can be administered directly.

An ultrasonic nebulizer has a large volume and can atomize a water-soluble drug into tiny mist particles of between about 1 um and 5 um at normal temperature. A jet nebulizer includes a compressed air source and an atomizer. The compressed air is suddenly decompressed after passing through a narrow opening at high speed and a negative pressure is generated locally so that the drug-containing solution is sucked out of the container by a siphoning effect. When subject to high-speed air flow, the drug-containing solution is broken into small aerosol particles by collision. A mesh nebulizer includes a stainless steel mesh covered with micropores having a diameter of about 3 The number of micropores on the mesh, which is conical, with the cone bottom facing the liquid surface, exceeds about 1000. FIG. 1a depicts an ultrasonic nebulizer. FIG. 1b depicts a jet nebulizer. FIG. 1c depicts the pressure vibration element and micropores of a mesh nebulizer.

Olodaterol is a selective fast-acting β2-adrenergic receptor agonist, chemically known as, 6-hydroxy-8-[(1R)-1-hydroxy-2-{[1-(4-methoxyphenyl)-2-methylpropan-2-yl]amino}ethyl]-3,4-dihydro-2H-1,4-benzoxazin-3-one. Olodaterol exhibits high selectivity towards the β2-adrenergic receptor (abbreviated as beta 2-receptor), exhibits rapid onset of action, and has a long half-life (more than 12 h). The bronchiectatic activity of olodaterol can be maintained for 24 h.

Budesonide is a glucocorticoid with efficient local anti-inflammatory effect, it can strengthen the stability of endotheliocyte, smooth muscle cell and lysosome membrane, Immunosuppression reaction and the synthesis of reduction antibody, thus the release of the activity media such as histamine is reduced and active reduction, and can alleviate antigen-antibody in conjunction with time the enzymatic processes that excites, suppress the synthesis of bronchoconstriction material and release and alleviate the contractile response of smooth muscle.

### BRIEF DESCRIPTION OF THE DRAWINGS

FIGS. 1, 2, and 3 depict various nebulizers or components of a nebulizer. FIG. 1 depicts an ultrasonic nebulizer, FIG. 2 depicts a jet nebulizer, and FIG. 3 depicts the pressure vibration element and micropores of a mesh nebulizer.

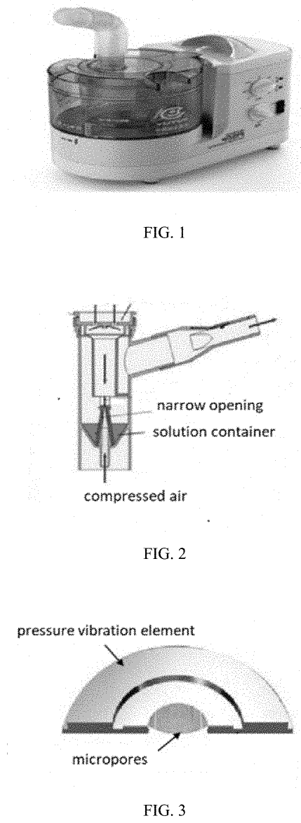

### EXAMPLES

Materials and reagents:
50% benzalkonium chloride aqueous solution purchased from Merck.
Edetate disodium dihydrate purchased from Merck.
Sodium hydroxide purchased from Titan reagents.
Hydrochloric acid purchased from Titan reagents.
Citric acid (CA) purchased from Merck.
Sodium chloride purchased from Merck.
Sulfobutylether β-cyclodextrin purchased from Zhiyuan Bio-tech Co., Ltd., China.
Budesonide and olodaterol purchased from SBECD purchased from Zhiyuan Bio-tech Co., Ltd., China.

### Example 1

The formulation and preparation of a solution for administration by nebulization inhalation (samples 1-4) is as follows:
1. Weigh the prescribed amount of SBECD in a beaker and add the prescribed amount of water to make the weight up to 104.70 g.
2. After stirring to dissolve SBECD, use CA to adjust the pH to about 4.00.
3. After adjusting the pH, add the prescribed amount of BD according to Table 1, and stir overnight in the dark to dissolve.
4. After the BD is dissolved, add the prescribed amount of OH according to Table 1, and filter after dissolving.
5. Divide the above samples into four parts, adjust the pH of each sample to 5.5, 5.0, 4.5, and 4.0, respectively, with CA solution. Designate the samples as Sample 1, Sample 2, Sample 3, and Sample 4, respectively.

TABLE 1
Ingredient of Samples 1-4

| Ingredients | Sample 1 | | Sample 2 | | Sample 3 | | Sample 4 | |
|---|---|---|---|---|---|---|---|---|
| Budesonide(BD) | 53.33 | mg | 53.33 | mg | 53.33 | mg | 53.33 | mg |
| Olodaterol | 1.842 | mg | 1.842 | mg | 1.842 | mg | 1.842 | mg |
| hydrochloride (OH) | | | | | | | | |
| Sulfobutyl ether β- | 10.00 | g | 10.00 | g | 10.00 | g | 10.00 | g |
| Cyclodextrin sodium | | | | | | | | |
| (SBECD) | | | | | | | | |

| | | | | |
|---|---|---|---|---|
| Citric acid (CA) | Adjusted to pH | Adjusted to pH | Adjusted to pH | Adjusted to pH |
| | 5.5 | 5.0 | 4.5 | 4.0 |
| Purified water | Added to | Added to | Added to | Added to |
| | 104.70 g | 104.70 g | 104.70 g | 104.70 g |

### Example 2

The thermal stability of samples 1˜4 of example 1 at 60° C. is provided below:

Impurity detection method:
Mobile phase A: accurately weigh 3.17 g of sodium dihydrogen phosphate, add 1 L of pure water to dissolve, adjust the pH of the solution to 3.20 with phosphoric acid. Mobile Phase B: Acetonitrile.
Instrument ID: HPLC.
Column ID: YMC-Triart C18 250*4.6 5 μm.
Detection wavelength: 230 nm.
Flow rate: 1.0 mL/min.
Running time: 35 min.

| Time | Mobile phase A % | Mobile Phase B % |
|---|---|---|
| 0 | 70 | 30 |
| 7 | 70 | 30 |
| 8 | 60 | 40 |
| 32 | 60 | 40 |
| 33 | 70 | 30 |
| 35 | 70 | 30 |

The test results are shown below.

TABLE 2
Thermal Stability of Samples 1-4 of Example 1at 60° C.
(Condition: 60° C. ± 2° C./RH 75%)

| | | | Sample 1 | Sample 2 | Sample 3 | Sample 4 |
|---|---|---|---|---|---|---|
| Ingredients | | | pH 5.5 | pH 5.0 | pH 4.5 | pH 4.0 |
| 0 day | Character | | Colorless clear liquid | | | |
| | Content μg/ml | OH | 17.87 | 18 | 17.94 | 17.98 |
| | | BD | 538.52 | 541.57 | 539.53 | 544.6 |
| | Total impurities % | OH | NA | 0.09 | 0.08 | 0.07 |
| | | BD | NA | NA | NA | NA |
| 60° C. | character | | Colorless clear liquid | | | |
| 7 days | Content μg/ml | OH | 17.33 | 16.98 | 17.13 | 17.22 |
| | | BD | 514.01 | 518.38 | 520.44 | 519.77 |
| | Total impurities % | OH | 2.6 | 2.16 | 2.94 | 2.17 |
| | | BD | 1.22 | 0.58 | 0.49 | 0.6 |
| 60° C. | Character | | Colorless clear liquid | | | |
| 14 days | Content μg/ml | OH | 17.09 | 17.3 | 17.64 | 16.48 |
| | | BD | 498.22 | 532.95 | 543.68 | 487.37 |
| | Total impurities % | OH | 2.62 | 7.17 | 4.08 | 3.13 |
| | | BD | 2.62 | 1.92 | 0.89 | 1.05 |

CA citric acid solution was used to adjust the pH. It was found that OH was relatively stable at pH 5.5 and pH 4.0, and BD was relatively stable at pH 4.0 and 4.5. Therefore, a formulation is relatively stable when adjusted to a pH of about 4.0 with citric acid.

### Example 3

The formulation and preparation of a solution for administration by nebulization inhalation (samples 5-8) is as follows:
1: Weigh the prescribed amount of SBECD into an empty beaker, add 93 g of pure water to dissolve, and adjust the pH to the target value with hydrochloric acid after dissolving.
2: Weigh the prescribed amount of BD into the above solution and stir in the dark until dissolved.
3: After dissolving, add OH into the above-solution until dissolved.
4: Adjust the pH of each sample to 5.0, 4.5, 4.0, and 3.5, respectively, with hydrochloric acid. Designate the samples as Sample 5, Sample 6, Sample 7, and Sample 8, respectively.
5: Add purified water to a final volume of 104.7 g.
6: Divide each Sample into parts and store at 40° C. and 60° C. for 5 days and 10 days. At each time point detect impurities.

TABLE 3
Ingredients of Samples 5-8

| Ingredients | Sample 5 | | Sample 6 | | Sample 7 | | Sample 8 | |
|---|---|---|---|---|---|---|---|---|
| Budesonide (BD) | 53.33 | mg | 53.33 | mg | 53.33 | mg | 53.33 | mg |
| Olodaterol | 1.842 | mg | 1.842 | mg | 1.842 | mg | 1.842 | mg |
| hydrochloride (OH) | | | | | | | | |
| Sulfobutyl ether β- | 10.00 | g | 10.00 | g | 10.00 | g | 10.00 | g |
| cyclodextrin sodium | | | | | | | | |
| (SBECD) | | | | | | | | |

| | | | | |
|---|---|---|---|---|
| HCl | Adjusted to pH | Adjusted to pH | Adjusted to pH | Adjusted to pH |
| | 5.0 | 4.5 | 4.0 | 3.5 |
| Purified water | Added to | Added to | Added to | Added to |
| | 104.70 g | 104.70 g | 104.70 g | 104.70 g |

TABLE 4
Thermal Stability of Samples 5-8 of Example 3 at 40° C.
(Condition: 40° C. ± 2° C./RH 75%)

| | | | Sample 5 | Sample 6 | Sample 7 | Sample 8 |
|---|---|---|---|---|---|---|
| Ingredients | | | pH 5.0 | pH 4.5 | pH 4.0 | pH 3.5 |
| 0 day | Character | | Colorless clear liquid | | | |
| | Total impurities % | OH | NA | NA | NA | NA |
| | | BD | 0.63 | 0.64 | 0.63 | 0.64 |
| 40° C. | Character | | Colorless clear liquid | | | |
| 5 days | Total impurities % | OH | 0.11 | 0.05 | 0.14 | 0.25 |
| | | BD | 0.97 | 0.98 | 0.98 | 0.98 |
| 40° C. | Character | | Colorless clear liquid | | | |
| 10 days | Total impurities % | OH | 0.14 | 0.41 | 1.03 | 1.79 |
| | | BD | 1.36 | 1.09 | 1.12 | 1.14 |

TABLE 5
Thermal Stability of Samples 5-8 of Example 3 at 60° C.
(Condition: 60° C. ± 2° C./RH 75%)

| | | | Sample 5 | Sample 6 | Sample 7 | Sample 8 |
|---|---|---|---|---|---|---|
| Ingredients | | | pH 5.0 | pH 4.5 | pH 4.0 | pH 3.5 |
| 0 day | Character | | Colorless clear liquid | | | |
| | Total impurities % | OH | 0.19 | 0.19 | 0.19 | 0.29 |
| | | BD | 0.63 | 0.64 | 0.63 | 0.64 |
| 60° C. | Character | | Colorless clear liquid | | | |
| 5 days | Total impurities % | OH | 0.59 | 0.62 | 0.94 | 1.71 |
| | | BD | 1.47 | 1.44 | 1.53 | 1.72 |
| 60° C. | Character | | Colorless clear liquid | | | |
| 10 days | Total impurities % | OH | 2.86 | 4.23 | 8.2 | 15.75 |
| | | BD | 1.8 | 1.66 | 1.68 | 1.92 |

A hydrochloric acid solution was used to adjust the pH. The total impurities after storage are as shown in the above table. At each different pH, each active ingredient exhibited different degrees of degradation. When using hydrochloric acid to adjust the pH, pH 5.0 exhibits the best stability at 60° C.±2° C./RH 75%

### Example 4

Solubility study to investigate the solubilizing effect of SBECD and Tween 80 on BD, and to investigate the solubility of BD at different concentrations.

To investigation the solubility of BD under different concentrations of SBECD: Weigh 0.1 g, 0.3 g, 0.5 g, and 1.0 g of BD into a 10 ml EP tube, add 10 ml of pure water, shake until it is completely dissolved, add excess BD (about 500 mg/100 mL), and wrap the EP tube in tin foil to protect from light. After being protected from light, place the EP tube on a shaker and shake for 24 hours.

To investigation of the solubility of BD under different concentrations of Tween 80: Weigh 0.002 g, 0.001 g, 0.0005 g, 0.1 g, 0.3 g, 0.5 g, and 1.0 g of BD into a weighing bottle, transfer the BD to a 10 ml EP tube by rinsing the weighing bottle with sufficient water to provide 10 ml in the EP tube, add excess BD (about 500 mg/100 mL), and wrap the EP tube in tin foil to protect it from light. After being protected from light, place the EP tube on a shaker and shake for 24 hours, then centrifuge to get the supernatant.

TABLE 6
Solubility of BD in Solutions Having Different Concentrations
of SBECD and Different Concentrations of Tween 80

| SBECD | BD Solubility | Tween 80 | BD Solubility |
|---|---|---|---|
| concentration | (mg/100 ml) | concentration | (mg/100 ml) |
| 1% | 16.91 | 1% | 16.54 |
| 3% | 40.28 | 3% | 38.88 |
| 5% | 61.91 | 5% | 64.84 |
| 10% | 81.3 | 10% | 118.79 |
| | | 5 mg/100 ml | 0.224 |
| | | 10 mg/100 ml | 0.256 |
| | | 20 mg/100 ml | 0.292 |

According to the above results, it can see that SBECD and Tween 80 have similar solubilization effects on BD. Tween-80 is within acceptable limits. According to the US pharmacopoeia, the concentration of Tween-80 should not exceed 20 mg/100 ml in an inhalation suspension. The solubility of BD in Tween-80 concentrations of 20 mg/100 ml is only 2.92 μg/ml. Unable to meet the requirements. A BD solubility of about 500 μg/ml is needed.

### Example 5

Aerodynamic Particle Size Distribution:

The aerodynamic particle size distribution of Sample 2 of Example 1 was determined using a Next Generation Pharmaceutical Impactor (NGI).

The device used to form the aerosol was a PARI E-flow device, purchased from PARI. The device was held close to the NGI inlet until no aerosol was visible. The flow rate of the NGI was set to 15 L/minute and was operated under ambient temperature and a relative humidity (RH) of 90%.

The solution of sample 2 was discharged into the NGI. Fractions of the dose were deposited at different stages of the NGI, in accordance with the particle size of the fraction. Each fraction was washed from the stage and analyzed using HPLC.

The result are shown in Table 7.

TABLE 7
Aerodynamic Particle Size Distribution of Sample 2

| | OH | | BD | | Cut-off |
|---|---|---|---|---|---|
| | | Percentage | | Percentage | diameters |
| | Dosage | content at all | Dosage | content at all | at 15 L/min |
| Deposited | (μg) | levels % | (μg) | levels % | (μm) |
| Device | 0.58 | 11.58 | 18.28 | 12.08 | |
| Throat | 0 | 0 | 1.2 | 0.79 | |
| Stage 1 | 0.06 | 1.20 | 2.41 | 1.59 | 14.10 |
| Stage 2 | 0.11 | 2.20 | 3.03 | 2.00 | 8.61 |
| Stage 3 | 0.47 | 9.38 | 13.84 | 9.15 | 5.39 |
| Stage 4 | 1.63 | 32.53 | 47.77 | 31.57 | 3.30 |
| Stage 5 | 1.40 | 27.94 | 40.78 | 26.95 | 2.08 |
| Stage 6 | 0.57 | 11.38 | 16.93 | 11.19 | 1.36 |
| Stage 7 | 0.18 | 3.59 | 5.24 | 3.46 | 0.98 |
| MOC | 0 | 0 | 0 | 0 | |
| Stage F | 0.01 | 0.20 | 1.85 | 1.22 | |

| | | |
|---|---|---|
| Theoretical dose (μg) | 5.56 | 161.25 |
| Actual test dose (μg) | 5.01 | 151.33 |
| Recovery rate (%) | 90.11 | 93.85 |
| MMAD (μm) | 3.33 | 3.33 |
| GSD | 1.57 | 1.58 |
| ISM (μg) | 4.37 | 129.44 |
| FPD (μg) | 3.79 | 112.57 |
| FPF (%) | 75.65 | 74.39 |

MOC is Micro-Orifice Collector.
ISM is Impactor Size Mass.
FPF is Fine Particle Fraction.
FPD is fine particle dose.
MMAD is mass median aerodynamic diameter.
GSD is Geometric Standard Deviation.
Stage F is a filter, which is a DDU tube connected to the end of the NGI.

### Comparative Example 1

The aerodynamic Particle Size Distribution of a budesonide suspension (Comparative Sample 1 (Pulmicort): batch number: LOT 324439; dosage: 0.5 mg; Specification: 2 ml/inhalation/time).

The budesonide suspension sample was purchased from AstraZeneca Pty Ltd.

The aerodynamic particle size distribution was determined using a Next Generation Pharmaceutical Impactor (NGI). The Sample was Pulmicort. The device used to form the aerosol was an LC-Plus, purchased from PARI in Germany. The device was held close to the NGI inlet until no aerosol was visible. The flow rate of the NGI was set to 30 L/minute and was operated under ambient temperature and a relative humidity (RH) of 90%.

The solution of Comparative Sample 1 was discharged into the NGI. Fractions of the dose were deposited at different stages of the NGI, in accordance with the particle size of the fraction. Each fraction was washed from the stage and analyzed using HPLC.

The result are shown in Table 8.

TABLE 8
Aerodynamic Particle Size Distribution of Budesonide
Suspension Comparative Sample 1 (Pulmicort)

| | BD | | |
|---|---|---|---|
| | | Percentage | Cut-off |
| | | content at | diameters at |
| Deposited | Dosage (μg) | all levels % | 30 L/min (μm) |
| Device | 892.42 | 91.86 | |
| Throat | 8.38 | 0.86 | |
| S1 | 6.98 | 0.72 | 11.72 |
| S2 | 16.71 | 1.72 | 6.40 |
| S3 | 22.43 | 2.31 | 3.99 |
| S4 | 17.25 | 1.78 | 2.30 |
| S5 | 5.15 | 0.53 | 1.36 |
| S6 | 2.15 | 0.22 | 0.83 |
| S7 | 0 | 0.00 | 0.54 |
| MOC | 0 | 0.00 | |

| | |
|---|---|
| Theoretical dose (μg) | 1000 |
| Actual test dose (μg) | 971.474 |
| Recovery rate (%) | 97.15 |
| ISM (μg) | 63.69 |
| FPD (μg) | 46.98 |
| FPF (%) | 4.84 |

A comparison of the NGI parameters for the budesonide suspension, Comparative Sample 1 (Pulmicort), and Sample 2 of Example 1 of the invention, shows that the effective lung deposition of Sample 2 of Example 1 is much higher than that of Comparative Example 1 (Pulmicort), indicating that the bioavailability of Sample 2 of Example 1 sprayed with the E-flow device is higher.

Because the ISM of Sample 2 of Example 1 is much higher than that of the Comparative Sample 1 (Pulmicort), in order to be consistent with the Pulmicort dose, it is considered that the effective dose of OH and BD can be reduced. Accordingly, with the formulation of the invention, the dose of OH is about 5.56 μg and the dose of BD is about 161.25 μg. Administering a lower dose can reduce the side effects of drugs on the human body.

### Comparative Example 2

Aerodynamic Particle Size Distribution of a budesonide inhalation aerosol

Comparative Sample 2: a budesonide suspension for inhalation was purchased from AstraZeneca Pty Ltd.

The budesonide suspension sample (Comparative Example 2) purchased from AstraZeneca Pty Ltd. administered 160 ug/press, contained 120 press/bottle, and was for administration by 2 presses/time, twice/day.

The aerodynamic particle size distribution was determined using a Next Generation Pharmaceutical Impactor (NGI). The Sample is Comparative Sample 2. The device used to nebulize the sample was an e-flow, purchased from PARI in Germany. The device was held close to the NGI inlet until no aerosol was visible. The flow rate of the NGI was set to 30 L/minute and the NGI was operated under ambient temperature and a relative humidity (RH) of 90%.

The solution of Comparative sample 2 was discharged into the NGI. Fractions of the dose were deposited at different stages of the NGI, in accordance with the particle size of the fraction. Each fraction was washed from the stage and analyzed using HPLC.

The result are shown in Table 9.

TABLE 9
Aerodynamic Particle Size Distribution of Comparative Sample 2

| | | Percentage | Cut-off |
|---|---|---|---|
| | | content at | diameter at |
| BD Deposited | Dosage (μg) | all levels % | 30 L/min (μm) |
| Device | 24.79 | 8.09 | |
| Throat | 116.32 | 37.95 | |
| Stage 1 | 4.8 | 1.57 | 11.72 |
| Stage 2 | 17.41 | 5.68 | 6.40 |
| Stage 3 | 64.66 | 21.09 | 3.99 |
| Stage 4 | 65.59 | 21.40 | 2.30 |
| Stage 5 | 12.96 | 4.23 | 1.36 |
| Stage 6 | 0 | 0 | 0.83 |
| Stage 7 | 0 | 0 | 0.54 |
| MOC | 0 | 0 | |
| Theoretical dose (μg) | | 320 | |

| | |
|---|---|
| Actual test dose (μg) | 306.53 |
| Recovery rate (%) | 95.79 |
| ISM | 160.62 μg |
| FPD | 143.21 μg |
| FPF | 46.72% |

The effective lung deposition of Sample 2 of Example 1 is much higher than that of Comparative Sample 2.

### Example 6

1: Weigh the prescribed amount of SBECD and NaCl into an empty beaker, add 93 g of pure water to dissolve, and adjust to the target pH with hydrochloric acid or CA according to Table 10.
2: Weigh the prescribed amount of BD into the above solution and dissolve.
3: After dissolving, add OH into the above solution and dissolve.
4: Adjust the pH of each sample to the target pH with hydrochloric acid or CA according to Table 10 and designate the samples as Sample 9 and Sample 10 respectively.
5: Add purified to a final weight of 104.70 g.

TABLE 10
Ingredients of Samples 9-10

| | | |
|---|---|---|
| Ingredients | Sample 9 | Sample 10 |

| | | | | |
|---|---|---|---|---|
| Budesonide (BD) | 53.33 | mg | 53.33 | mg |
| Olodaterol | 1.842 | mg | 1.842 | mg |
| hydrochloride (OH) | | | | |
| Sulfobutyl ether β- | 10.00 | g | 10.00 | g |
| cyclodextrin sodium | | | | |
| (SBECD) | | | | |
| NaCl | 100 | mg | 100 | mg |

| | | |
|---|---|---|
| Adjusted pH | Adjusted to pH | Adjusted to pH |
| | 5.0 with HCl | 4.0 with CA |
| Purified water | Added to | Added to |
| | 104.70 g | 104.70 g |

1. A liquid, propellant-free pharmaceutical formulation comprising:
(a) budesonide and olodaterol;
(b) a solvent; and
(c) a pharmacologically acceptable solubilizing agent;
wherein the solubilizing agent is selected from the group consisting of a cyclodextrin derivative or a salt thereof, and combinations thereof.
wherein the pharmaceutical formulation has a pH ranging from about 2.0 to about 6.0.

2. The pharmaceutical formulation according to claim 1, wherein budesonide is present in an amount ranging from about 1 mcg/ml to about 100 mcg/ml.

3. The pharmaceutical formulation according to claim 1, wherein the olodaterol is present in an amount ranging from about 2 mcg/ml to about 500 mcg/ml.

4. The pharmaceutical formulation according to claim 1, wherein the solvent is a water substantially free of other solvents.

5. The pharmaceutical formulation according to claim 1, wherein the solubilizing agent is sulfobutylether β-cyclodextrin sodium.

6. The pharmaceutical formulation according to claim 5, wherein the solubilizing agent is present in an amount ranging from about 1 g/100 ml to about 40 g/100 ml.

7. The pharmaceutical formulation according to claim 5, wherein the solubilizing agent is present in an amount ranging from about 0.04 g/4 ml to about 1.6 g/4 ml.

8. The pharmaceutical formulation according to claim 1, further comprising a pharmacologically acceptable preservative selected from the group consisting of benzalkonium chloride, benzoic acid, and sodium benzoate.

9. The pharmaceutical formulation according to claim 8, wherein the pharmacologically acceptable preservative is present in an amount ranging from about 0.08 mg/4 ml to about 12 mg/4 ml.

10. The pharmaceutical formulation according to claim 8, wherein the pharmacologically acceptable preservative is benzalkonium chloride in an amount of about 0.4 mg/4 ml.

11. The pharmaceutical formulation according to claim 1, further comprising a stabilizer selected from the group consisting of edetic acid (EDTA), edetate disodium, edetate disodium dihydrate, and citric acid, and wherein the stabilizer is present in an amount ranging from about 0.04 mg/4 ml to about 20 mg/4 ml.

12. The pharmaceutical formulation according to claim 1, further comprising sodium chloride in an amount ranging from about 0.1 g/100 ml to about 0.9 g/100 ml.

13. A method for administering the pharmaceutical formulation according to claim 1, comprising nebulizing a defined amount of the pharmaceutical formulation with an inhaler by using pressure to force the pharmaceutical formulation through a nozzle to form an inhalable aerosol.

14. The method according to claim 13, wherein the defined amount of the pharmaceutical formulation is less than about 8 microliters of the pharmaceutical formulation.

15. The method according to claim 13, wherein the average particle size of the aerosol is less than about 15 micron.

16. A method of treating asthma or COPD in a patient, comprising administering to the patient the pharmaceutical formulation according to claim 1.

17. The method of claim 16, wherein the pharmaceutical formulation is administered at a therapeutically effective dose of budesonide ranging from about 1 μg to about 100 μg and a therapeutically effective dose of olodaterol ranging from about 5 μg to about 500 μg.

18. The pharmaceutical formulation according to claim 1, wherein budesonide is present in an amount of about 500 μg/mL.

19. The liquid, propellant-free pharmaceutical formulation of claim 1 comprising:
an aqueous solution of:
(a) budesonide in an amount of about 1 μg/mL to about 1000 m/mL;
(b) olodaterol in an amount of about 2 μg/mL to about 500 m/mL;
(c) sulfobutyl ether β-cyclodextrin sodium in an amount of about 1 g/mL to about 40 g/100 ml;
(d) sodium chloride in an amount of about 0.1 g/100 mL to about 0.9 g/100 mL; and
(e) citric acid in an amount sufficient to adjust the pH to about 4.0.

20. The liquid, propellant-free pharmaceutical formulation of claim 1 comprising:
an aqueous solution of:
(a) budesonide in an amount of about 1 μg/mL to about 1000 μg/mL;
(b) olodaterol in an amount of about 2 μg/mL to about 500 μg/mL;
(c) sulfobutyl ether β-cyclodextrin sodium in an amount of about 1 g/mL to about 40 g/100 mL;
(d) sodium chloride in an amount of about 0.1 g/100 mL to about 0.9 g/100 mL; and
(e) hydrochloric acid in an amount sufficient to adjust the pH to about 5.0.

21. The liquid, propellant-free pharmaceutical formulation of claim 1 comprising:
an aqueous solution of:
(a) budesonide in an amount of about 50.9 mg/100 mL;
(b) olodaterol in an amount of about 1.8 mg/100 mL;
(c) sulfobutyl ether β-cyclodextrin sodium in an amount of about 9.6 mg/mL; and
(d) citric acid in an amount sufficient to adjust the pH to about 4.0

22. The liquid, propellant-free pharmaceutical formulation of claim 1 comprising:
an aqueous solution of:
(a) budesonide in an amount of about 50.9 mg/100 mL;
(b) olodaterol in an amount of about 1.8 mg/100 mL;
(c) sulfobutyl ether β-cyclodextrin sodium in an amount of about 9.6 mg/mL; and
(d) hydrochloric acid in an amount sufficient to adjust the pH to about 5.0
